(19) United States
(12) Patent Application Publication
(10) Pub. No.: US 2025/0366833 A1
(43) Pub. Date: Dec. 4, 2025

(54) SYSTEM AND METHOD TO DETERMINE WEIGHT OF BOWEL MOVEMENT AND LOG DATA

(71) Applicant: Michael Warren, Baltimore, MD (US)

(72) Inventor: Michael Warren, Baltimore, MD (US)

(21) Appl. No.: 19/195,736

(22) Filed: May 1, 2025

Related U.S. Application Data
(60) Provisional application No. 63/652,708, filed on May 29, 2024.

(51) Int. Cl. A61B 10/00; E03D 11/00; G01G 19/52; G01G 23/37

(52) U.S. Cl. CPC A61B 10/0038; E03D 11/00; G01G 19/52; G01G 23/3742

(57) ABSTRACT

A system and method for a weight monitoring system attached to a toilet, whereby one or more force sensors positioned on the underside of a toilet seat of a toilet measure weight before and after a session on the toilet such that the data may be sent to a mobile application for tracking losses and gains.

### CROSS REFERENCE TO RELATED APPLICATIONS

This application claims priority to U.S. Provisional Patent Application No. 63/652,708 filed on May 29, 2024.

### FIELD OF THE DISCLOSURE

The overall field of this invention relates to weight scales for identifying user weight and more particularly to one or more force sensors connected to a toilet seat capable of receiving data and sending the data to a mobile computing device for analysis.

### BACKGROUND

Monitoring weight regularly has been increasingly popular with users. Regularly tracking weight helps individuals keep tabs on their general health. Sudden weight gain or loss can be an indicator of underlying health issues such as hormonal imbalances, metabolic disorders, or other medical conditions. Other people also like to measure their weight for fun such as after going to the bathroom. Scales to measure weight are a frequent fixture in many homes, often found in bathrooms, where it is used to measure weight and other health metrics. However, scales can be cumbersome and pose safety risks, particularly in confined spaces like bathrooms. To prevent accidents such as tripping or collisions with the bathroom door, many people store their scales under furniture or in bathroom corners. This practice, while reducing the risk of accidents, can be inconvenient and even dangerous, particularly for individuals with limited mobility or muscle strength. Another method has been to have a toilet seat with a built-in scale but then the user has to buy a new toilet or toilet seat.

### SUMMARY

The present invention is directed to a system and method for weight determination for a toilet seat or other device. The system has one or more force sensors for turning weight readings into signals; a microcontroller for automatically collecting and transmitting weight data; a software module for identifying; a communication module to enable transmission of data to a database; a battery or other power system for powering the unit; and a cloud database for analyzing weight, duration, and frequency data as well as sharing aforementioned data.

In some aspects, the techniques described herein relate to a weight monitoring system, including: one or more force sensors positioned on the underside of a toilet seat of a toilet to measure weight before and after a session on the toilet.

In some aspects, the techniques described herein relate to a weight monitoring system, wherein a user can be positively identified by the system via by mechanical interface, electronic interface, magnetic interface, or combination of interfaces thereof, such as mobile phone or RFID tag, registered to the user on a platform and/or paired with a device.

In some aspects, the techniques described herein relate to a weight monitoring system for a toilet, including: one or more force sensors attached to an underside of a toilet seat; a control system including: a microcontroller operatively connected to the one or more force sensors; an analog-to-digital converter configured to convert signals from the one or more force sensors into digital data; a memory storing computer-readable instructions; and a wireless communication module, wherein the control system is configured to: receive force measurements from the one or more force sensors; calculate a weight value based on the force measurements; and transmit the weight value to a remote computing device.

In some aspects, the techniques described herein relate to a weight monitoring system, further including: a sensor configured to detect whether the toilet seat lid is in an open position or a closed position; and wherein the control system is further configured to initiate weight measurements when the sensor detects the toilet seat lid is in the open position.

In some aspects, the techniques described herein relate to a weight monitoring system, wherein the sensor includes at least one of: an infrared detector; a magnetic sensor; an ultrasonic sensor; or a mechanical switch.

In some aspects, the techniques described herein relate to a weight monitoring system, wherein the one or more force sensors are configured to be attached to the toilet seat as a substitute for toilet seat bumpers

In some aspects, the techniques described herein relate to a weight monitoring system, further including: a mobile application configured to: receive the weight value from the control system; store the weight value in association with a timestamp; display the weight value to a user; and track weight changes over time.

In some aspects, the techniques described herein relate to a weight monitoring system, wherein the mobile application is further configured to: compare weight values before and after toilet use; calculate weight differences between measurements; generate alerts when weight changes exceed predetermined thresholds; and display historical weight data.

In some aspects, the techniques described herein relate to a weight monitoring system, wherein the one or more force sensors include: at least three separate force sensing units positioned at different points under the toilet seat; and wherein the force sensing units are connected to each other through at least one of: a wired connection or a wireless connection using a local area network protocol.

In some aspects, the techniques described herein relate to a weight monitoring system, wherein the control system is further configured to: enter a low-power mode after a predetermined period of inactivity; automatically wake from the low-power mode when the one or more force sensors detect applied pressure; and resume normal operation upon waking.

In some aspects, the techniques described herein relate to a weight monitoring system, wherein the mobile application is further configured to: synchronize weight data with an external calendar; display weight measurements in relation to user activities; and allow users to customize measurement schedules based on daily routines.

In some aspects, the techniques described herein relate to a weight monitoring system, further including: an imaging system configured to: capture images during toilet use; analyze the images using machine learning algorithms; identify characteristics of waste material; and generate health-related insights based on the analysis.

In some aspects, the techniques described herein relate to a weight monitoring system, wherein the machine learning algorithms include: pre-trained neural networks configured for transfer learning; pattern recognition capabilities; and predictive modeling functions for health condition identification.

In some aspects, the techniques described herein relate to the weight monitoring system, wherein the control system is further configured to: calibrate the one or more force sensors upon initial installation; compensate for temperature variations affecting sensor readings; detect and correct for sensor drift over time; and provide accuracy indicators for weight measurements.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure.

FIG. 1 shows a block diagram of the various systems of the weight monitoring system.

FIG. 2 shows a block diagram of the communication system of the weight monitoring system.

FIG. 3 shows a block diagram of the computing devices of weight monitoring system.

FIG. 4 shows an illustration of the weight monitoring system.

FIG. 5 shows an illustration of the spacers.

### DETAILED DESCRIPTION

The present invention is directed to discrete devices consisting of a lightweight electronic enclosure and force sensors. The electronics include an analog to digital converter (ADC) and microcontroller. The one or more force sensors can be affixed (such as by adhesive, mechanical clip, etc.) to the underside of any toilet seat and housed in one or more bumpers typically used to separate the toilet seat from the toilet base. The device may be connected to a mobile application for monitoring weight, analyzing stool, and comparing with other users. The device may also identify whether the lid is open and/or closed via an electrical mechanism, mechanical mechanism, magnetic mechanism, or any combination thereof.

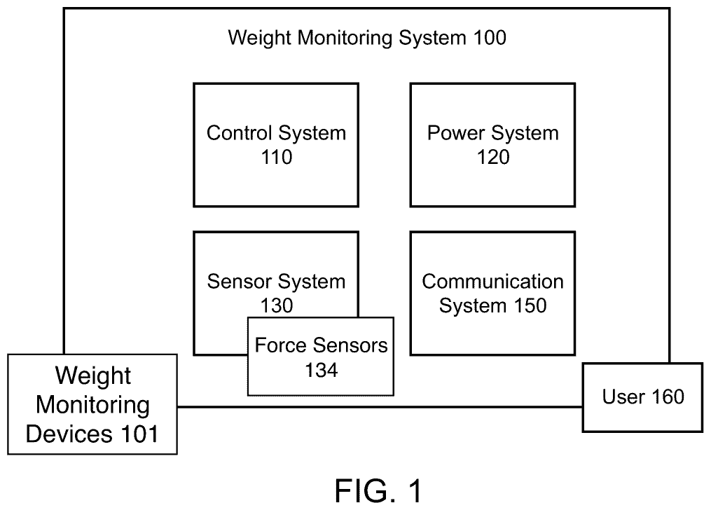

With reference now to FIG. 1, FIG. 1 illustrates a block diagram of an exemplary embodiment of weight monitoring system 100 with one or more weight monitoring devices. Weight monitoring system 100 may have a plurality of subsystems including a control system 110, a power system 120, a sensor system 130, a communication system 150, which may be integrated into the overall system and structure of weight monitoring system 100 via wired or wireless connection. The various systems may be individually configured and correlated with respect to each other so as to attain the desired objective of measuring the user's weight.

Power system 120 of weight monitoring system 100 provides the power to the circuits and components of the sensor system 130, control system 110, and communication system 150 during the process of measuring the weight of a user or during other operations performed by the system. Weight monitoring system 100 may be powered by methods known by those of ordinary skill in the art. In some embodiments, weight monitoring system 100 may plug into an electrical outlet using an electrical cord to supply power. Further, weight monitoring system 100 may include a rechargeable battery pack or disposable battery receptacle whereby the rechargeable battery is of a charge, design, and capacity, to provide sufficient power to weight monitoring system 100 and its subsystems for a suitable period of time. In other non-limiting embodiments weight monitoring system 100 may contain subcomponents that work together or separately, each with their own power system 120 and integrated wirelessly.

Control system 110 may operate to control the actuation of the other systems. Control system 110 may have a series of computing devices which will be discussed in detail later in the description. Control system 110 may be in the form of a circuit board, a memory or other non-transient storage medium in which computer-readable coded instructions are stored and one or more processors configured to execute the instructions stored in the memory. Control system 110 may have a wireless transmitter, a wireless receiver, and a related computer process executing on the processors.

Computing devices 200 of control system 110, may be any type of computing device that typically operates under the control of one or more operating systems, which control scheduling of tasks and access to system resources. Computing devices 200 may be a Single Board Computer (SBC), such as Raspberry Pi®, or other computing devices such as but not limited to a phone, tablet, television, desktop computer, laptop computer, gaming system, wearable device electronic glasses, networked router, networked switch, networked, bridge, or any computing device capable of executing instructions with sufficient processor power and memory capacity to perform operations of control system 110.

The one or more computing devices 200 may be integrated into control system 110, while in other non-limiting embodiments, control system 110 may be a remotely located computing device or server configured to communicate with one or more other control systems 110. Control system 110 may also include an internet connection, network connection, and/or other wired or wireless means of communication (e.g., LAN, etc.) to interact with other components. The connection allows a user, such as user 160, to update, control, send/retrieve information, monitor or otherwise interact passively or actively with control system 110.

Control system 110 may include control circuitry and one or more microprocessors or controllers acting as a servo control mechanism capable of receiving input from sensor system 130 and communication system 150, analyzing the input from sensor system 130 and communication system 150, and generating an output signal to communication system 150 and power system 120. The microprocessors (not shown) may have on-board memory to control the power that is applied to power system 120, and communication system 150 in response to input signals from the user 160 and from sensor system 130.

Control system 110 may include circuitry to provide an actuable interface for user 160 to interact with, including switches and indicators and accompanying circuitry for an electronic control panel, mechanical control panel, or magnetic control panel. Control system 110 may be preprogrammed with any reference values, by any combination of hardwiring, software, firmware to implement various operational modes including but not limited to temperature, light, and humidity values, whereby the system may calibrate the one or more force sensors upon initial installation and compensate for temperature, light, and humidity variations affecting sensor readings and correct for sensor drift over time for more accurate measurements.

The microprocessors in control system 110 may also monitor the current state of circuitry within control system 110 to determine the specific mode of operation chosen by the user. For instance, when “on”, the microprocessors may begin autonomously determining weight at predetermined intervals. Further, such microprocessors that may be part of control system 110 may receive signals from any of or all systems, including without limitation, power system 120 and communication system 150.

Control system 110 may include an analog-to-digital converter (ADC) that converts continuous signals, such as sound, pressure, or light, from the analog form into digital data. The ADC receives an analog input, a continuous signal that can vary across a potentially infinite range of values. The ADC samples the continuous analog signal at discrete intervals of time. The frequency at which the ADC samples the analog signal is known as the sampling rate. Each sample is then quantized, meaning it is approximated by a finite set of values (usually represented as binary numbers). During this process, small errors called quantization errors can occur, as the values are rounded to the nearest available level. The final output from an ADC is a digital numeric value that approximates the original analog input. This digital output can then be processed, stored, or transmitted by digital systems.

Sensor system 130, as shown in FIG. 1, may include a plurality of detectors mounted or otherwise connected to control system 110. Sensor system 130 may have one or more force sensors or additional pressure sensors or mechanical switches positioned on the toilet seat or inside the housing of weight monitoring devices 101. Force sensors 134 may have mechanism for sensing force such as mechanical switches that are activated when enough force is applied to the plate, strain gauges that can measure the exact amount of force applied, piezoelectric sensors that generate a voltage when pressure is applied, or Force Sensing Resistors (FSRs) that decrease in resistance when pressure is applied, which can then be measured to determine the force.

When pressure is applied to the toilet seat, force sensors 134 generate a signal and sensor system 130 sends the signal to control system 110. Control system 110 may receive this signal from sensor system 130, whereby the microprocessors then compare the received input value from sensor system 130 using a comparison function and are able to evaluate the input data against a setting or expectation of a certain reference value stored within the memory of control system 110. Control system 110 determines the weight currently being applied to the toilet seat. In some embodiments, control system 110 may also receive a signal from communication system 150 that user 160 wishes to use weight monitoring system 100.

In one non-limiting embodiment, sensor system 130 may have infrared (“IR”) detectors having photodiode and related amplification and detection circuitry to sense the opening and closing of the toilet lid such that force sensors 134 may automatically (or after a predetermined period of time) begin to calculate data once the toilet lid is determined to be open. Further, this embodiment may be used to discern whether the toilet lid is open or closed when weight is applied. In other embodiments, radio frequencies, magnetic fields, and ultrasonic sensors, temperature sensors, pressure sensors, humidity sensors, or other types of sensors and transducers may be employed. Detectors may be arranged in any number of configurations and arrangements.

Sensor system 130 may further include environmental adaptation mechanisms may include water-level sensing capabilities using ultrasonic or optical sensors to account for water displacement factors in weight calculations. Humidity and temperature sensors with corresponding compensation algorithms may maintain measurement accuracy across varying bathroom environments. Atmospheric pressure sensors may adjust calculations in different elevation conditions or weather changes that might affect measurement precision. Self-calibration routines may automatically adjust for long-term environmental changes, ensuring consistent measurement accuracy without requiring manual intervention. Machine learning algorithms may identify and compensate for household-specific environmental patterns that could influence measurement accuracy.

Control system 110 may receive this signal from sensor system 130, whereby the microprocessors then compare the received input value from sensor system 130 using a comparison function and are able to evaluate the input data against a setting or expectation of a certain reference value stored within the memory of control system 110. Control system 110 determines that the toilet seat lid is currently open or closed. Sensor system 130 may also include a video imaging module having a HD video imaging element (e.g., camera) and associated electronic hardware to wirelessly send acquired video and images to computing device 200.

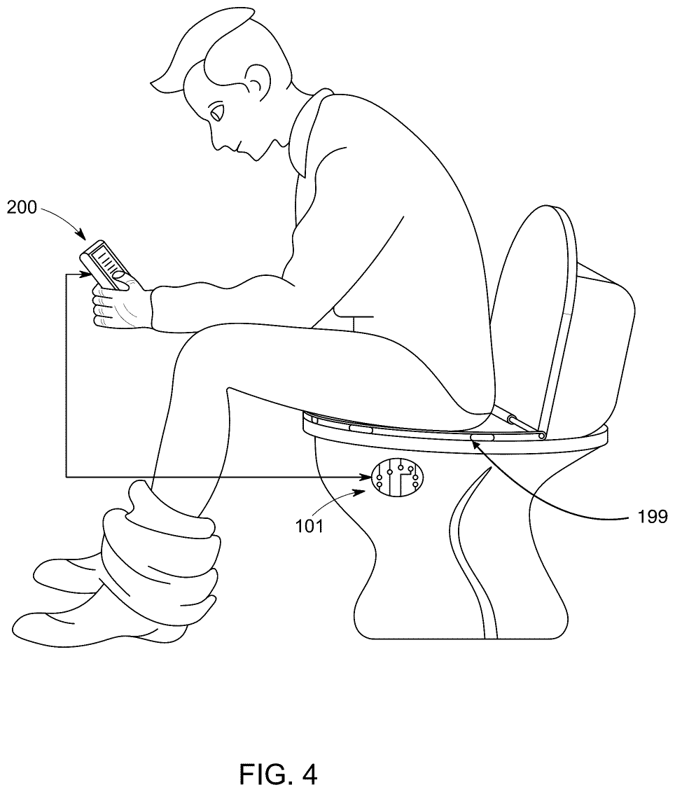

Turning to FIG. 4, FIG. 4 shows an illustration of a weight monitoring device 101 for measuring weight before and after a bathroom session on the toilet. Weight monitoring device 101 may have a housing with a hollow frame and a base. In one or more non-limiting embodiments, weight monitoring device 101 may be in the form of a series of bumpers 199 or spacers positioned on the underside of a toilet seat to provide a buffer or gap between the toilet seat and the bowl itself. Bumpers 199 may be affixed by any number of fasteners such as but not limited to adhesive, clips, latches, hinges, buckles, or magnets to the underside of any toilet seat such that they may be removably positioned to retrofit an existing toilet.

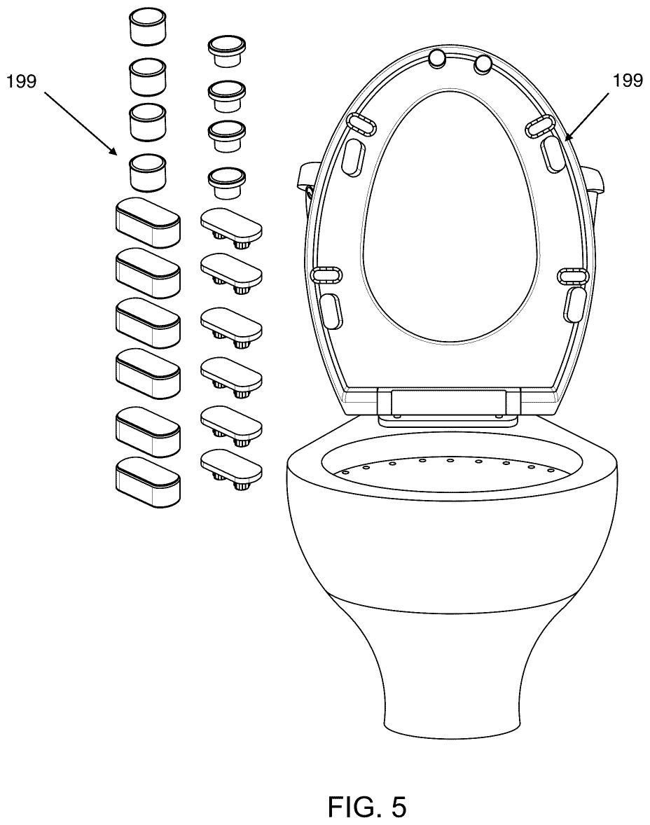

The bumpers 199 may be connected to each other by a series of cords or wirelessly through a local area network such as Bluetooth® or other wireless connections known by those of ordinary skill in the art including but not limited to a mesh network. In other non-limiting embodiments weight monitoring devices 101 may be built into the toilet seat or the toilet. FIG. 5 illustrates various types of bumpers that may be used and where they may be positioned on the underside of the toilet seat.

Bumpers 199 may include modular component architecture allowing selective upgrade or replacement of individual sensors without complete system replacement. Medical-grade waterproof housing with antimicrobial surface treatments may be used to prevent pathogen colonization. Bumpers 199 may include self-cleaning mechanisms for optical sensors that may use hydrophobic coatings and automated cleaning routines. Bumpers 199 may include impact-resistant construction that may withstand falls and collisions common in bathroom environments. Bumpers 199 may include thermal management systems that may maintain optimal operating temperatures for sensitive electronic components despite humidity and temperature fluctuations characteristic of bathroom environments.

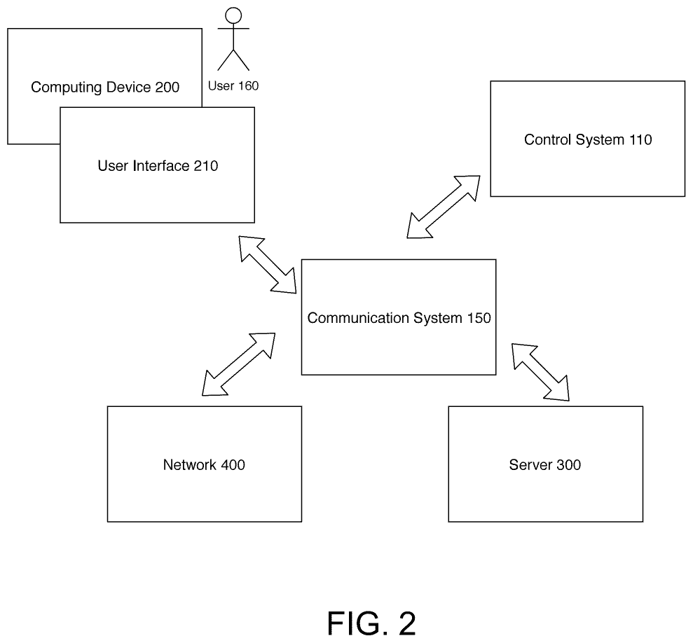

Turning to FIG. 2, FIG. 2 shows an exemplary block diagram of a communication system of weight monitoring system 100. Control system 110 may be in communication with communication system 150, as illustrated in FIG. 2, to connect with other computing devices whereby signals transmitted from the computing devices may be received by control system 110. Communication system 150 may interact with control system 110 using computing device 200 to associate data with user 160 and allow user 160 to interact with control system 110 using a computing device 200. User 160 may access a user interface, such as user interface 210 using computing device 200. User interface 210 may have a plurality of buttons or icons that are selectable by user 160 for communication system 150 to perform particular processes in response to the selections. In one or more non-limiting embodiments, communication system 150 may be innate, built into, or otherwise integrated into existing platforms or systems such as a website, a third-party program, Apple™ operating systems (e.g., iOS), Android™, Snapchat™, Instagram™, Facebook™, or any other platform.

Computing device 200 of communication system 150 may be similar to the computing devices of control system 110 and may be any type of computing device that typically operates under the control of one or more operating systems, which control scheduling of tasks and access to system resources. Computing device 200, may in some embodiments, be a computing device such as an iPhone™, Android-based™ phone, or Windows-based™ phone, a tablet, television, desktop computer, laptop computer, gaming system, wearable device electronic glasses, networked router, networked switch, networked, bridge, or any computing device capable of executing instructions with sufficient processor power and memory capacity to perform operations of weight monitoring system 100 while in communication with network.

In some embodiments, computing devices 200 may be in communication with one or more servers 300 such as server 300 via communication system 150 or one or more networks such as network 400 connected to communication system 150. Server 300 may be located at a data center or any other location suitable for providing service to network 400 whereby server 300 may be in one central location or in many different locations in multiple arrangements including on the cloud. Server 300 may comprise a database server such as MySQL® or Maria DB® server. Server 300 may have an attached data storage system storing software applications and data. Server 300 has a number of modules that provide various functions related to communication system 150. Modules may be in the form of software or computer programs that interact with the operating system of server 300 whereby data such as weight gains and losses over predetermined time periods is collected in databases for storage as instruction-based expressions of components and/or processes under communication system 150 and may be processed by one or more processors within server 300 or another component of communication system 150 as well as in conjunction with execution of one or more other computer programs.

Modules may be configured to receive commands or requests from computing devices 200, server 300, and outside connected devices over network 400. Server 300 may comprise components, subsystems and modules to support one or more management services for weight monitoring system 100.

In one or more non-limiting embodiments, network 400 may include a local area network (LAN), such as a company Intranet, a metropolitan area network (MAN), or a wide area network (WAN), such as the Internet or World Wide Web. Network 400 may be a private network or a public network, or a combination thereof. Network 400 may be any type of network known in the art, including telecommunications network, a wireless network (including Wi-Fi), and a wireline network. Network 400 may include mobile telephone networks utilizing any protocol or protocols used to communicate among mobile digital computing devices (e.g., computing device 200), such as GSM, GPRS, UMTS, AMPS, TDMA, or CDMA. In one or more non-limiting embodiments, different types of data may be transmitted via network 400 via different protocols. In alternative embodiments, computing devices 200, may act as standalone devices or whereby they may operate as peer machines in a peer-to-peer (or distributed) network environment.

Network 400 may further include a system of terminals, gateways, and routers. Network 400 may employ one or more cellular access technologies including 2nd (2G), 3rd (3G), 4th (4G), 5th (5G), LTE, Global System for Mobile communication (GSM), General Packet Radio Services (GPRS), Enhanced Data GSM Environment (EDGE), and other access technologies that may provide for broader coverage between computing devices if, for instance, they are in a remote location not accessible by other networks.

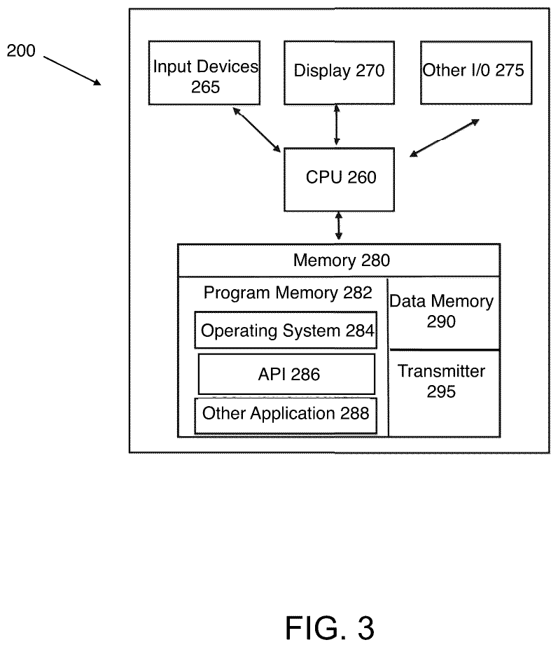

Turning to FIG. 3, FIG. 3 is a block diagram showing various components of computing device 200. Computing device 200 may comprise a housing for containing one or more hardware components that allow access to edit and query communication system 150. Computing device 200 may include one or more input devices such as input devices 265 that provide input to a CPU (processor) such as CPU 260 of actions related to user 160. Input devices 265 may be implemented as a keyboard, a touchscreen, a mouse, via voice activation, wearable input device, a camera, a trackball, a microphone, a fingerprint reader, an infrared port, a controller, a remote control, a fax machine, and combinations thereof.

Actions may be initiated by a hardware controller that interprets the signals received from input device 265 and communicates the information to CPU 260 using a communication protocol. CPU 260 may be a single processing unit or multiple processing units in a device or distributed across multiple devices. CPU 260 may be coupled to other hardware devices, such as one or more memory devices with the use of a bus, such as a PCI bus or SCSI bus. CPU 260 may communicate with a hardware controller for devices, such as for a display 270. Display 270 may be used to display text and graphics. In some examples, display 270 provides graphical and textual visual feedback to a user.

In one or more embodiments, display 270 may include an input device 265 as part of display 270, such as when input device 265 is a touchscreen or is equipped with an eye direction monitoring system. In some implementations, display 270 is separate from input device 265. Examples of display 270 include but are not limited to: an LCD display screen, an LED display screen, a projected, holographic, virtual reality display, or augmented reality display (such as a heads-up display device or a head-mounted device), wearable device electronic glasses, contact lenses capable of computer-generated sensory input and displaying data, and so on. Display 270 may also comprise a touch screen interface operable to detect and receive touch input such as a tap or a swiping gesture. Other I/O devices such as I/O devices 275 may also be coupled to the processor, such as a network card, video card, audio card, USB, FireWire or other external device, camera, printer, speakers, CD-ROM drive, DVD drive, disk drive, or Blu-Ray device. In further non-limiting embodiments, a display may be used as an output device, such as, but not limited to, a computer monitor, a speaker, a television, a smart phone, a fax machine, a printer, or combinations thereof.

CPU 260 may have access to a memory such as memory 280. Memory 280 may include one or more of various hardware devices for volatile and non-volatile storage and may include both read-only and writable memory. For example, memory 280 may comprise random access memory (RAM), CPU registers, read-only memory (ROM), and writable non-volatile memory, such as flash memory, hard drives, floppy disks, CDs, DVDs, magnetic storage devices, tape drives, device buffers, and so forth. Memory 280 may be a non-transitory memory.

Memory 280 may include program memory such as program memory 282 capable of storing programs and software, including an operating system, such as operating system 284. Memory 280 may further include an application programing interface (API), such as API 286, and other computerized programs or application programs such as application programs 288. Memory 280 may also include data memory such as data memory 290 that may include database query results, configuration data, settings, user options, user preferences, or other types of data, which may be provided to program memory 282 or any element of computing devices 200.

Computing device 200 may have a transmitter 295, such as transmitter 295, to transmit data. Transmitter 295 may have a wired or wireless connection and may comprise a multi-band cellular transmitter to connect to the server 300 over 2G/3G/4G cellular networks. Other embodiments may also utilize Near Field Communication (NFC), Bluetooth, or another method to communicate information.

Users 160 may initially register to become a registered user 160 associated with weight monitoring system 100. Weight monitoring system 100 may be downloadable and installable on computing device 200. In one or more non-limiting embodiments, communication system 150 may be preinstalled on computing devices 200 by the manufacturer or designer. Further, communication system 150 may be implemented using a web browser via a browser extension or plugin. Server 300 may associate computing devices 200 with an account during the registration process. The account may be user 160 specific or specific to a home, enclosure, or toilet, whereby a unique identification of the entity may be stored in the account.

Upon successful authentication of user 160, a homepage or dashboard may be generated. The homepage may be modified, deleted, written to, or otherwise administered by the respective user 160. Display 270 may be presented to user 160 through user interface 210 that may comprise a number of different subpages viewable or accessible through user interface 210 by selecting one or more tabs.

User interface 210 on computing device 200 may display statuses for multiple entities such as multiple weight monitoring devices 101 placed on the underside of a toilet. Accordingly, one area may have multiple systems under multiple toilets, each of which may be separately controllable and viewable on a user's computing device 200. For example, user interface 210 may display information to user 160 logged into an account that includes two different weight monitoring devices 101 that may be located in a master bathroom and a guest bathroom. A status for each weight monitoring device 101 may be displayed on a list or another format on a user's computing device 200 (e.g., including a smartphone). In one embodiment, the list may be a dynamic list based on weight or other criteria.

Different states of weight monitoring devices 101 may be displayed by different indicators through user interface 210, whereby control system 110 receives data from sensor system 130, analyzes the data, and presents the data in the form of indicators to user 160 through user interface 210 of communication system 150. For example, user interface 210 may display a weight before a predetermined time period and after a predetermined time period with a linear graph at different intervals such as one minute. User interface 210 may display colors for passing certain thresholds or gaining or losing weight. The colors are for illustrative purposes only, as one of ordinary skills would understand alternative colors or shapes or forms of indicating these statuses (including via text and words) may be utilized instead.

User interface 210 may have an adjustable timer component for each weight monitoring device 101 to operate in, whereby the timer component may enable input from user 160 for control system 110 to start calculating data determining a predetermined time period. In one embodiment, user interface 210 may allow user 160 to receive data at certain times of the day. For instance, user interface 210 may present to users 160 options to switch the state of control system 110 to operate at preprogrammed times, at times determined according to a random pattern, or any other variation. User interface 210 may present one or more clocks that provide an understanding of time of day, month, and year that was weighed.

User interface 210 may further include a calendar or be synchronized with an outside calendar to display user's 160 activities and weight gains and losses such as how much weight was lost during a toilet use and over a set period of time such as a day, whereby control system 110 may be customized through user interface 210 to be active or inactive during these user activities. If user 160 has multiple weight monitoring devices 101, user 160 may be presented with the option to specify which weight monitoring devices as well as inactivating or preventing certain weight monitoring devices 101 from displaying data. Calendars may have different indicators such as color, shapes, font, or change in appearance to distinguish the different occurrences and the different weight monitoring devices 101 from one another. In one or more non-limiting embodiments, user 160 may select among multiple templates, designs, or formats in which appointments or events may be presented. User interface 210 may provide user 160 with options to share calendars between accounts as well as other users such that the system may generate comparative statistics across the multiple users that have connected with the user through the mobile application for competitions or friendly rivalries.

In other embodiments, control system 110 may have an energy saver mode, whereby user interface 210 may allow user 160 to switch control system 110 to an off or hibernation state. Further, control system 110 may automatically turn off or enter a hibernation state at a particular time of day or after an elapsed amount of time based on predefined parameters.

User interface 210 may display messages for events generated by weight monitoring system 100 such as when a new record has been met for the most amount of weight lost during a bathroom session. All of the messages for events that occur with weight monitoring system 100 may be grouped into a single thread to organize the messages. In a similar manner, user interface 210 may display the history of events for weight monitoring system 100.

In one or more non-limiting embodiments, weight monitoring system 100 may analyze stool samples using the video feed and then apply transfer learning to develop deep learning models. In transfer learning, the neural network is trained in two stages. The first stage may include pre-training, where the network is generally trained on a large-scale benchmark dataset representing a wide diversity of labels/categories. The second stage may include fine-tuning, where the pretrained network is further trained on the specific target task of interest, which may have fewer labeled examples than the pre training dataset. The pre-training step helps the network learn general features that can be reused on the target task. For training, the machine learning algorithm system will use a set of input images to identify the image properties that, when used, will result in the correct classification of the image and also for predictive modeling, whereby once the system has learned how to classify images, the learned model maybe applied to new images to assist in identifying a disease or disease progression or other problem in the stool sample.

Weight monitoring systems then may utilize machine learning that uses shared data and/or predictive modeling to identify and/or predict problems with the stool sample in images. The system may include, without limitation thereto, the VIM device, the VIM software application, AI, and pattern recognition. The system may also include one or more imaging devices coupled to one or more processors, whereby the one or more processors obtain training data from one or more first images, wherein one or more abnormal regions and one or more normal regions of the stool are identified. The system may then receive a second image captured by one or more cameras at a later time than the one or more first images and/or captured using a different imaging technique, and generate, using machine learning trained using the training data, one or more viewable indicators identifying one or more abnormalities of the stool sample in the second image and alert or display the analysis to user 160 on user interface 210. In further embodiments, weight monitoring system 100 may detect abnormal weight change patterns based on predetermined thresholds of collected user data whereby weight monitoring system 100 may generate automated alerts when the abnormal patterns are detected and provide recommendations for health monitoring based on the detected patterns.

The weight monitoring system may include a multi-user identification system with biometric recognition capabilities integrated directly into the toilet seat sensors. Artificial intelligence algorithms may identify users based on unique sitting posture and pressure distribution patterns. The system may integrate with existing smart home ecosystems for automatic user profile selection and data management. RFID readers positioned within the housing may detect user-specific RFID tags, enabling automatic profile selection without manual interaction. Machine learning algorithms may analyze historical usage patterns to improve identification accuracy over time, distinguishing between multiple household members with high precision after sufficient training periods

The system may incorporate advanced health analytics capabilities that correlate bowel movement metrics with dietary information from connected nutrition tracking applications. Integration protocols may incorporate additional health metrics from wearable devices and smart home sensors for comprehensive health insights. Predictive modeling using machine learning may identify potential digestive health issues before symptomatic onset. The analytics system may include seasonal pattern recognition to establish personalized baselines accounting for normal dietary variations throughout the year. Comparative analysis against anonymized population data may contextualize individual measurements within demographic-appropriate norms, with adjustable privacy settings allowing users to control data sharing granularity.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention.

The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. A weight monitoring system, comprising: one or more bumpers positioned on an underside of a toilet seat of a toilet to measure weight before and after a session on the toilet, wherein the one or more bumpers separate the toilet seat from the toilet, wherein the one or more bumpers have one or more force sensors to measure the weight before and after the session on the toilet.

2. The weight monitoring system of claim 1, further comprising a mobile application on a device, wherein a user is positively identified by the system via by mechanical interface, electronic interface, magnetic interface, or combination of interfaces thereof, such as mobile phone or RFID tag, registered to the user on a platform or paired with the device.

3. A weight monitoring system for a toilet, comprising:
one or more force sensors attached to an underside of a toilet seat;
a control system comprising: a microcontroller operatively connected to the one or more force sensors;
an analog-to-digital converter configured to convert signals from the one or more force sensors into data;
a memory storing computer-readable instructions; and
a wireless communication module, wherein the control system is configured to: receive force measurements from the one or more force sensors; calculate a weight value based on the force measurements; and transmit the weight value to a remote computing device.

4. The weight monitoring system of claim 3, wherein the one or more force sensors are configured to be attached to the toilet seat as a substitute for toilet seat bumpers.

5. The weight monitoring system of claim 3, wherein the one or more force sensors are configured as part of an enclosure or enclosures to be attached to the toilet seat as a substitute for toilet seat bumpers.

6. The weight monitoring system of claim 3, further comprising: a mobile application configured to: receive the weight value from the control system; store the weight value in association with a timestamp; display the weight value to a user; and track weight changes over time.
